(19) United States
(12) Patent Application Publication
(10) Pub. No.: US 2004/0242638 A1
(43) Pub. Date: Dec. 2, 2004

(54) Use of an h1 antagonist and a safe steroid to treat rhinitis

(76) Inventors: John M Yanni, Burleson, TX (US); Daniel A Gamache, Arlington, TX (US)

Correspondence Address: Alcon Research, 6201 South Freeway, Fort Worth, TX, 76134-2099, US

(21) Appl. No.: 10/497,619

(22) PCT Filed: Nov. 18, 2002
(86) PCT No.: PCT/US02/36915

Related U.S. Application Data
(60) Provisional application No. 60/337,371, filed on Dec. 5, 2001.

(51) Int. Cl. A61K 31/473; A61K 31/454

(52) U.S. Cl. 514/326; 514/290

(57) ABSTRACT

Compositions and methods for treating rhinitis with H₁ antagonists/antiallergics and safe steroids are disclosed.

The present invention is directed to the use of an H₁ antagonist/antiallergic in combination with a safe steroid to treat nasal conditions, specifically rhinitis.

### BACKGROUND OF THE INVENTION

Allergic rhinitis has historically been treated with a regimen of oral antihistamines and/or oral steroids. Systemic treatment typically requires higher concentrations of the drug compound to be administered to afford an effective concentration to reach the necessary treatment site. Antihistamine compounds are known to have central nervous system (CNS) activity which manifests itself in drowsiness. They may also have anticholinergic activity which manifests itself in the drying of mucus membranes. Steroid therapy whether dosed orally or intranasally can also produce significant systemic side effects, including adrenal insufficiency, cardiovascular irregularities, and immunosuppression. Growth retardation is an especially important concern in allergic pediatric patents.

Intranasal combination therapy is known. For example, WO 97/01337 discloses combinations of topical nasal antihistamines and topical nasal steroids for the treatment of rhinitis. It does not disclose the use of the safe steroids of the present invention. WO 97/46243 discloses a nasal spray containing a steroid and an antihistamine. This publication does not disclose or suggest the use of a safe steroid. There are also intranasal products containing both a steroid and an antihistamine, among other active ingredients, (e.g., Cortinasal from Pharmacobel; Neowine from Dupa; Nicorin from Rontag; Rinosular from SmithKline Beecham; Rinocusi from Cusi; and Comfonin from Meider.)

The use of an H₁ antagonist/antiallergic in combination with a safe steroid for treating rhinitis is not known.

### SUMMARY OF THE INVENTION

The present invention is directed to intranasal compositions of combinations of H₁ antagonists/antiallergic and safe steroids to treat rhinitis. Methods for the use of the compositions in mammals are also contemplated.

### DESCRIPTION OF PREFERRED EMBODIMENTS

The current invention comprises compositions of H₁ antagonists/antiallergics for treating the sneezing and rhinorrhea associated with allergic rhinitis. The compositions also include a safe steroid, as used herein the term “safe steroids” means a steroid which treats eosinophil and neurotrophil associated inflammation with resultant congestion but has either a lack of systemic bioavailability or is rapidly deactivated after systemic absorption.

The H₁ antagonists/antiallergics which are useful according to the present invention include all efficacious compounds, including, but not limited to: emedastine, loratadine, 5-[2-[4-bis(4-fluorophenyl)hydroxymethyl-1-piperidinyl]ethyl]-3-methyl]-2-oxazolidinone ethanedioate), desloratadine, azelastine, olopatadine, levocabastine, epinastine, and ketotifen.

Safe steroids which can be used herein include any glucocorticoid which meets the safe steroid definition, including but not limited to, rimexolone and loteprednol.

The H₁ antagonists/antiallergics and safe steroids (compounds) can be incorporated into various types of intranasal formulations for delivery to the nose. For example, intranasal formulations may contain preservatives, such as, benzalkonium chloride, EDTA, and tromethamine; viscosity modifiers, such as, hydroxy propyl methyl cellulose (HPMC) and related agents; toxicity adjusting agents, for example, sodium chloride (NaCl); wetting agents/surfactants, such as, tyloxapol or Polysorbate 80; pH adjusters; and water.

The compounds are preferably formulated as intranasal suspensions or solutions, with a pH of about 6.0 to 8.0. The H₁ antagonists/antiallergics will normally be contained in these formulations in an amount 0.01% to 0.5% by weight, but preferably in an amount of 0.02% to 0.1% by weight. The safe steroids will normally be contained in those formulations in an amount 0.05% to 1.5% by weight, but preferably in an amount of 0.1% to 1.0% by weight. Thus, for intranasal presentation 1 to 2 sprays of these formulations would be delivered to the nostrils up to 2 times per day according to the routine discretion of a skilled clinician.

The preferred compositions of the present invention includes olopatadine (0.1%) with rimexolone (0.1%) and emedastine 0.05% with rimexolone (0.1%).

The following example is illustrative of a composition of the present invention, but is in no way limiting.

### EXAMPLE

| Ingredient | Weight % |
|---|---|
| Emedastine | 0.05% |
| Rimexolone | 0.1% |
| Benzalkonium chloride | 0.01% |
| Tromethamine | 0.5% |
| Disodium EDTA | 0.01% |
| Sodium Chloride (Adjust isotonicity to | 0.6 to 0.8% |
| 280 mOsmols/kg) | |
| HPMC | 0.1 to 0.5% |
| Tyloxapol | 0.05% |
| NaOH and/or HCl | QS to pH 7.4 |
| Purified water | QS to 100% |

We claim:

1. A method of treating rhinitis in mammals which comprises administering a pharmaceutically effective amount of a composition comprising an H₁ antagonist/antiallergic and a safe steroid.

2. The method of claim 1 wherein the composition comprises an H₁ antagonist/antiallergic selected from the group consisting of emedastine, loratadine, 5-[2-[4-bis(4-fluorophenyl)hydroxymethyl-1-piperidinyl]ethyl]-3-methyl]-2-oxazolidinone ethanedioate), desloratadine, azelastine, olopatadine, levocabastine, epinastine, and ketotifen.

3. The method of claim 1 wherein the composition comprises a safe steroid selected from the group consisting of rimexolone and loteprednol.

4. The method of claim 2 wherein the composition comprises an antagonist/antiallergic selected from the group consisting of emedastine, olopatadine, and desloratadine.

5. A method of treating rhinitis in mammals which comprises administering a pharmaceutically effective amount of a composition comprising emedastine and rimexolone.

6. A method of treating rhinitis in mammals which comprises administering a pharmaceutically effective amount of a composition comprising olopatadine and rimexolone.

7. A method of treating rhinitis in mammals which comprises administering a pharmaceutically effective amount of a composition comprising desloratadine and rimexolone.
